(19) United States
(12) Patent Application Publication
(10) Pub. No.: US 2022/0410131 A1
(43) Pub. Date: Dec. 29, 2022

(54) ALKYLAROMATIC CONVERSION CATALYST SYSTEM

(71) Applicant: SHELL USA, INC., HOUSTON, Texas, TX (US)

(72) Inventors: Rikeshchandra Sharadchandra JOSHI, Bangalore (IN); Roy VAN DEN BERG, Amsterdam (NL); Erik ZUIDEMA, Amsterdam (NL); Yuriy Ihorovych YANSON, Rotterdam (NL)

(21) Appl. No.: 17/787,636

(22) PCT Filed: Dec. 21, 2020
(86) PCT No.: PCT/EP2020/087433

§ 371 Date: Jun. 21, 2022

(30) Foreign Application Priority Data
Dec. 23, 2019 (EP) 19219400.9

(51) Int. Cl. B01J 29/40; B01J 37/00; B01J 35/00; B01J 35/02; C07C 4/18

(52) U.S. Cl. CPC B01J 29/40; B01J 37/0018; B01J 35/0006; B01J 35/023; C07C 4/18; B01J 2229/16; C07C 2529/40

(57) ABSTRACT

An alkylaromatic conversion catalyst system having (a) a first catalyst composition having (i) a carrier which includes a binder composition prepared from a mixture having one or more oligomerized alkoxy silicates and one or more hydrolyzing agents; and a ZSM-5 zeolite; (ii) one or more metals chosen from the group consisting of Groups 6, 9, 10 and 11; and optionally, (iii) a Group 14 metal; and (b) a second catalyst composition having (i) a carrier which includes a refractory oxide binder and a zeolite selected from one or more of ZSM-5, ferrierite, ZSM-11, ZSM-12 and EU-1; (ii) one or more metals chosen from the group consisting of Groups 6, 9, 10 and 11; and optionally, (iii) a Group 14 metal.

The present invention relates to an alkylaromatic conversion catalyst system and its use in ethylbenzene dealkylation.

### BACKGROUND OF THE INVENTION

Ethylbenzene (EB) is one of the aromatic hydrocarbons that can be obtained from naphtha pyrolysis or reformate. Reformate is an aromatic product obtained by the catalyzed conversion of straight-run hydrocarbons boiling in the 70 to 190° C. range, such as straight-run naphtha. The catalysts used for the production of reformate are often platinum-on-alumina catalysts. On conversion to reformate, the aromatics content is considerably increased and the resulting hydrocarbon mixture becomes highly desirable as a source of valuable chemical intermediates and as a component for gasoline. The principle components are a group of aromatics often referred to as BTX: benzene, toluene and the xylenes, including ethylbenzene. Other components may be present such as their hydrogenated homologues, e.g. cyclohexane.

Of the BTX group, the most valuable components are benzene and the xylenes, and therefore BTX is often subjected to processing to increase the proportion of those two aromatics: hydrodealkylation of toluene to benzene and toluene disproportionation to benzene and xylenes. Within the xylenes, para-xylene is the most useful commodity and xylene isomerisation or transalkylation processes have been developed to increase the proportion of para-xylene.

A further process that can be applied is the hydrodealkylation of ethylbenzene to benzene.

Generally, it is preferred to isolate BTX from the reformate stream, then isolate the C₈ aromatics by distillation, followed by extraction of para-xylene via selective adsorption or crystallization. The para-xylene lean C₈ aromatics stream is then subjected to xylene isomerisation with the aim of maximising the para-xylene component to be able to recycle the stream and extract more para-xylene. To avoid build-up of ethylbenzene in the recycle stream, ethylbenzene has to be converted. Typically, this is done either by dealkylating ethylbenzene to generate valuable benzene or by reforming ethylbenzene to xylenes to increase the yield of xylenes. In practice, catalyst systems are used to isomerize the xylenes to equilibrium and simultaneously either reform ethylbenzene to xylenes or dealkylate ethylbenzene. The latter process is the subject of the present invention.

In ethylbenzene dealkylation, it is a primary concern to ensure not just a high degree of conversion of ethylbenzene to benzene and isomerize the xylenes close to equilibrium, but also to avoid xylene loss.

Xylenes may typically be lost due to transalkylation, e.g. between benzene and xylene to give toluene, or by addition of hydrogen to form, for example, alkenes or alkanes. A further route for xylene loss is the disproportionation of two xylene molecules, leading to the formation of the significantly less valuable trimethylbenzene (TMB) and toluene.

It is therefore the aim of the present invention to provide a catalyst that will convert ethylbenzene to benzene with a reduced xylene loss, and in particular, with a reduced TMB make.

For the conversion of BTX streams to increase the proportion of closely configured molecules, a wide range of proposals utilizing zeolitic catalysts have been made. One common zeolite group utilized in the dealkylation of ethylbenzene is the MFI zeolites and, in particular, ZSM-5. The ZSM-5 zeolite is well known and documented in the art.

US 2019/0232262 A1 provides a process for dealkylation of alkylaromatic compounds which process comprises contacting an alkylaromatic feedstock with i) a first catalyst comprising a) a carrier which comprises of from 20 to 70% by weight of a refractory oxide binder and of from 30 to 80 wt. % of dealuminated ZSM-5 having a crystallite size of from 500 to 10,000 nm and a silica to alumina molar ratio in the range of from 20 to 100; b) an amount of from 0.001 to 5 wt. % of one or more metals chosen from the group consisting of Groups 6, 9 and 10; and optionally c) a metal chosen from Group 14 in an amount up to 0.5 wt. %, and ii) a subsequent catalyst comprising a) a carrier which comprises of from 20 to 70 wt. % of a refractory oxide binder; of from 30 to 80 wt. % of ZSM-5 having a crystallite size of from 3 to 100 nm and a silica to alumina molar ratio in the range of from 20 to 200; b) an amount of from 0.001 to 5 wt. % of one or more metals chosen from the group consisting of Groups 6, 9 and 10; and optionally c) a metal chosen from Group 14 in an amount up to 0.5 wt. %.

Many preparation routes have been proposed that provide active MFI zeolites, including ZSM-5, see for example U.S. Pat. No. 3,702,886 A, references provided in the Atlas, or Database, of Zeolite Structures, and in other literature references such as by Yu et al. in Microporous and Mesoporous Materials 95 (2006) 234 to 240, and Iwayama et al. in U.S. Pat. No. 4,511,547 A.

U.S. Pat. No. 3,702,886 A prepares the zeolites utilizing a silica source, an alumina source and alkali sources and describes the use of a tetraalkylammonium cation, such as tetrapropylammonium (TPA) cation, as an organic structure-directing agent in the preparation of a ZSM-5.

U.S. Pat. No. 8,574,542 B2 describes the preparation of ZSM-5 by synthesis from an aqueous reaction mixture comprising an alumina source, a silica source, an alkali source and L-tartaric acid or a water-soluble salt thereof and the use of said ZSM-5 in a process for the conversion of an aromatic hydrocarbon-containing feedstock, in particular, for the selective dealkylation of ethylbenzene.

Catalysts manufactured for use in the dealkylation of ethylbenzene are typically required to undergo a passivation pre-treatment. However, such pre-treatments are complicated and costly and can dilute zeolite content and affect catalyst performance by causing pore blockages.

Therefore, whilst catalyst systems have been described in the art for the dealkylation of ethylbenzene, there is a continued need to develop catalyst systems which not only demonstrate high ethylbenzene conversion, but which also can be manufactured without the need for separate passivation pretreatments.

### SUMMARY OF THE INVENTION

The present invention provides an alkylaromatic conversion catalyst system comprising,

(a) a first catalyst composition comprising,
(i) a carrier which comprises in the range of from 5 to 70 wt. % of a binder composition prepared from a mixture comprising one or more oligomerized alkoxy silicates and one or more hydrolyzing agents; and in the range of from 30 to 95 wt. % of a ZSM-5 zeolite, based on the total weight of the carrier;
(ii) an amount in the range of from 0.001 to 5 wt. % of one or more metals chosen from the group consisting of Groups 6, 9, 10 and 11; and optionally,
(iii) up to 0.5 wt. % of a Group 14 metal, on the basis of the total first catalyst composition; and
(b) a second catalyst composition comprising,
(i) a carrier which comprises in the range of from 20 to 90 wt. % of refractory oxide binder and in the range of from 10 to 80 wt. % of zeolite selected from one or more of ZSM-5, ferrierite, ZSM-11, ZSM-12 and EU-1, based on the total weight of the carrier;
(ii) an amount in the range of from 0.001 to 5 wt. % of one or more metals chosen from the group consisting of Groups 6, 9, 10 and 11; and optionally,
(iii) up to 0.5 wt. % of a Group 14 metal, on the basis of the total second catalyst composition.

In another embodiment, the present invention provides a method of making said alkylaromatic conversion catalyst system, wherein said method comprises making the first catalyst composition by:—

(i) preparing a binder composition from a mixture comprising one or more oligomerized alkoxy silicates and one or more hydrolyzing agents;
(ii) preparing a carrier comprising in the range of from 5 to 70 wt. % of said binder composition; and in the range of from 30 to 95 wt. % of a ZSM-5 zeolite, based on the total weight of the carrier; and
(iii) depositing on the carrier an amount in the range of from 0.001 to 5 wt. % of one or more metals chosen from the group consisting of Groups 6, 9, 10 and 11; and optionally, up to 0.5 wt. % of a Group 14 metal, on the basis of the total first catalyst composition.

In yet another embodiment, the present invention provides an ethylbenzene dealkylation process which comprises contacting a hydrocarbon feedstock which comprises ethylbenzene in the presence of hydrogen with said alkylaromatic conversion catalyst system.

### DETAILED DESCRIPTION OF THE INVENTION

All weight amounts, as the term is used in relation with the catalyst composition or the catalyst preparation, are based on the basis of total catalyst and on dry amounts. Any water and other solvent present in the starting compounds are to be disregarded.

In addition to the metal dopants used therein, the carrier used in the first and second catalyst compositions is also catalytically active and plays an important role in the dealkylation of ethylbenzene.

The crystallite size is measured by Transmission Electron Microscopy (TEM) with the average based on the number average.

Groups 6, 9, 10, 11 and 14 are as defined in the IUPAC Periodic Table of Elements dated 1 May 2013.

The weight amounts of metal are calculated as amount of metal on total weight of catalyst independent of the actual form of the metal.

The bulk or overall SAR can be determined by any one of a number of chemical analysis techniques. Such techniques include X-ray fluorescence, atomic adsorption, and inductive coupled plasma-atomic emission spectroscopy (ICP-AES). All techniques will provide substantially the same bulk ratio value. The molar silica-to-alumina ratio for use in the present invention is preferably determined by X-ray fluorescence.

The ZSM-5 zeolite for use in the first catalyst composition preferably has a molar silica-to-alumina ratio (SAR) in the range of from 20 to 100. More preferably, the SAR of the ZSM-5 zeolite in the first catalyst composition is more preferably at least 25, most preferably at least 30, and is preferably at most 100, more preferably at most 90, most preferably at most 80, especially at most 70. In particular, the SAR of the ZSM-5 zeolite in the first catalyst composition is preferably in the range of from 50 to 80, more preferably in the range of from 50 to 70.

In one embodiment of the present invention, the ZSM-5 zeolite for use in the first catalyst composition is prepared by synthesis from an aqueous reaction mixture comprising one or more alumina sources, one or more silica sources, one or more alkali sources and L-tartaric acid or a water-soluble salt thereof. Full details on ZSM-5 zeolites which may be conveniently used in the present invention are described in U.S. Pat. No. 8,574,542 B2.

In a preferred embodiment of the present invention, the ZSM-5 zeolite for use in the first catalyst composition is prepared by synthesis from an aqueous reaction mixture comprising one or more alumina sources, one or more silica sources, one or more alkali sources, and one or more amines.

In a particularly preferred embodiment, the ZSM-5 zeolite for use in the first catalyst composition is prepared by synthesis from an aqueous reaction mixture comprising one or more alumina sources, one or more silica sources, one or more alkali sources, and one or more amines wherein said amines are primary and/or secondary amines.

The crystallite size of the ZSM-5 zeolite of the first catalyst composition is preferably in the range of from 200 to 5000 nm, more preferably in the range of from 1000 to 5000 nm.

The first catalyst composition preferably comprises ZSM-5 zeolite that has undergone a dealumination treatment, i.e. removal of alumina from the zeolite framework. Examples of dealumination treatments include steaming, treatment with F-containing salts (for example, as described in U.S. Pat. No. 4,753,910 A) and treatment with acids such as hydrochloric acid (HCl), nitric acid (HNO₃) or ethylenediamine tetraacetic acid (EDTA). Said dealumination treatment may have taken place either prior to mixing the ZSM-5 zeolite with the binder composition (i.e. on the ZSM-5 zeolite per se) or after extrusion of the carrier comprising said ZSM-5 zeolite and binder composition. In many cases, it is preferred to carry out the afore-mentioned treatment on the extrudates.

In one embodiment of the present invention, it is preferred to treat the ZSM-5 zeolite particles or extrudate by a steaming process comprising a heat treatment at temperatures above 300° C. in the presence of steam in order to remove alumina from the zeolite framework. The extent of dealumination depends on the steam concentration and the temperature. In a preferred embodiment, the temperature is in the range of from 500 to 750° C. and the steam concentration in air is in the range of from 10 to 25%.

The zeolite of the second catalyst composition may be selected from one or more of ZSM-5, ferrierite, ZSM-11, ZSM-12 and EU-1. Preferably, said zeolite comprises ZSM-5. More preferably, said zeolite is ZSM-5.

The zeolite of the second catalyst composition may also undergo a similar dealumination treatment as hereinbefore described in relation to the zeolite of the first catalyst composition. Such a treatment includes steaming, treatment with F-containing salts and treatment with acids such as hydrochloric acid (HCl), nitric acid (HNO₃) or ethylenediaminetetraacetic acid (EDTA). Such a treatment may take place either prior to mixing the zeolite with the binder composition (i.e. on the zeolite per se) or after extrusion of the carrier comprising said zeolite and binder composition. In many cases, it is preferred to carry out the afore-mentioned treatment on the extrudates. However, in a specific embodiment of the present invention, the zeolite of the second catalyst composition does not undergo a dealumination treatment.

The crystallite size of the zeolite of the second catalyst composition is preferably at least 3 nm, more specifically at least 5 nm, more specifically at least 10 nm, more specifically at least 20 nm. The crystallite size of the zeolite of the second catalyst preferably is at most 100 nm, more specifically at most 90 nm, more specifically at most 80 nm, more specifically at most 70 nm, more specifically at most 60 nm, more specifically at most 50 nm, most specifically at most 40 nm. In a preferred embodiment of the present invention, the zeolite of the second catalyst composition has a crystallite size in the range of from 3 to 100 nm.

The molar silica-to-alumina ratio of the zeolite of the second catalyst composition preferably is at least 20, more specifically at least 30, more specifically at least 40, most specifically at least 50. This ratio preferably is at most 200, more specifically at most 180, more specifically at most 150, more specifically at most 120 and most specifically at most 110. In a preferred embodiment of the present invention, the zeolite of the second catalyst composition has a SAR in the range of from 20 to 200.

In addition to providing an alkylaromatic conversion catalyst system as described herein, the present invention also provides a method of making said alkylaromatic conversion catalyst system. Said method comprises making the first catalyst composition by:—

(i) preparing a binder composition from a mixture comprising one or more oligomerized alkoxy silicates and one or more hydrolyzing agents;
(ii) preparing a carrier comprising in the range of from 5 to 70 wt. % of said binder composition; and in the range of from 30 to 95 wt. % of a ZSM-5 zeolite, based on the total weight of the carrier; and
(iii) depositing on the carrier an amount in the range of from 0.001 to 5 wt. % of one or more metals chosen from the group consisting of Groups 6, 9, 10 and 11; and optionally, up to 0.5 wt. % of a Group 14 metal, on the basis of the total first catalyst composition.

As described hereinbefore, the binder composition for use in the first catalyst composition is a suspension prepared from mixture of one or more oligomerized alkoxy silicates and one or more hydrolyzing agents.

By “alkoxy silicate” herein is meant a chemical compound with the formula Si(OR)₄ where R could be a linear alkyl group with 1 to 6 carbon atoms. Accordingly, by “oligomerized alkoxy silicates” is meant the hydrolyzed form of the corresponding alkoxy silicates which contains silyl ether (Si—O—Si) linkages. Said oligomerized alkoxy silicates may be linear, branched or cyclic oligomers.

In one embodiment, wherein n represents the number of silicate monomers and is in the range of from 2 to 8, the silicate monomers could be linked together in a linear, branched or cyclic way.

Preferably, the oligomerized alkoxy silicates have the following general formula:—

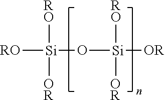

where n=1, 2, or higher and R is a linear alkyl group with 1 to 6 carbon atoms. Preferably R is ethyl group.

In a preferred embodiment, the oligomerized alkoxy silicates are ethyl silicates having the following general formula:—

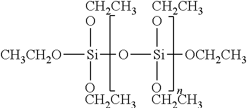

where n=1, 2, or higher. Preferably, n is in the range of from 1 to 12, more preferably in the range of 3 to 5.

Monomeric alkoxy silicates (for example, tetraethyl orthosilicate (TEOS)) (where n=0) is not suitable for use as a binder composition in the first catalyst composition.

Alkoxy silicates are typically prepared by alcoholysis of silicon tetrachloride and oligomerized alkoxy silicates are prepared by controlled hydrolysis/poly condensation reaction of alkoxy silicates. Chemically, oligomerized alkoxy silicates and alkoxy silicates are different and have different CAS nos. assigned.

Examples of materials comprising oligomerized alkoxy silicates that may be conveniently used to prepare the binder composition for use in the first catalyst composition include Ethyl Silicate 30 (available under the trade designation “Dynasylan Silbond HT-30” from Evonik), Ethyl silicate 33 (available under the trade designation “Dynasylan Silbond HT-33” from Evonik), Ethyl Silicate 40 (available under the CAS number [18954-71-7]), Ethyl Silicate 50 (available under the trade designation “Dynasylan Silbond 50” from Evonik).

Hydrolyzing agents that may be employed in the preparation of said binder composition include water, aqueous solutions of metal nitrate salts such as aluminium nitrate, aqueous and organic solutions of acids such as nitric acid, hydrochloric acid, sulfuric acid and phosphoric acid, and bases such as ammonia, urea, ethylenediamine, dipropylamine and other organic amine compounds. Preferred hydrolyzing agents are aqueous solutions of nitric acid or ammonia.

The carrier of the first catalyst composition comprises in the range of from 5 to 30 wt. % of the afore-mentioned binder composition and in the range of from 70 to 95 wt. % of a ZSM-5 zeolite, more specifically in the range of from 5 to 20 wt. % of the afore-mentioned binder composition in combination with in the range of from 80 to 95 wt. % of ZSM-5 zeolite and most specifically in the range of from 5 to 15 wt. % of the afore-mentioned binder composition in combination with in the range of from 85 to 95 wt. % of ZSM-5 zeolite, all based on the total weight of the carrier.

Whilst the carrier for use in the first catalyst composition is prepared by steps comprising:—

(i) preparing a binder composition from a mixture comprising one or more oligomerized alkoxy silicates and one or more hydrolyzing agents;
(ii) preparing a carrier comprising in the range of from 5 to 70 wt. % of said binder composition; and in the range of from 30 to 95 wt. % of a ZSM-5 zeolite, based on the total weight of the carrier, it will be appreciated that the binder composition may be prepared separately prior to mixing with the ZSM-5 zeolite or said binder composition may be prepared in situ from a mixture comprising one or more oligomerized alkoxy silicates, one or more hydrolyzing agents and the ZSM-5 zeolite. There is no limitation on the order in which the one or more oligomerized alkoxy silicates, one or more hydrolyzing agents and the ZSM-5 zeolite are mixed together during in situ preparation of the carrier. For example, the one or more oligomerized alkoxy silicates may be first mixed with the ZSM-5 zeolite prior to the addition of the one or more hydrolyzing agents or the one or more oligomerized alkoxy silicates may be first mixed with the one or more hydrolyzing agents prior to the addition of the ZSM-5 zeolite.

In a preferred embodiment of the method of making the alkylaromatic conversion catalyst system as described herein, the ZSM-5 zeolite in the first catalyst composition undergoes a dealumination treatment. Said dealumination treatment may take place on the ZSM-5 zeolite prior to use in the carrier. Alternatively, the carrier comprising the binder composition and ZSM-5 zeolite may undergo dealumination treatment. Said dealumination treatment preferably comprises steaming.

In one embodiment of the present invention, the carrier of the first catalyst composition may further comprise an amount of a refractory binder selected from alumina, silica-alumina, aluminium phosphate, silica, zirconia and titania.

In the method of making the alkylaromatic conversion catalyst system as described herein, the second catalyst composition may be made by:—

(i) preparing a carrier which comprises in the range of from 20 to 90 wt. % of refractory oxide binder and in the range of from 10 to 80 wt. % of zeolite selected from one or more of ZSM-5, ferrierite, ZSM-11, ZSM-12 and EU-1, based on the total weight of the carrier; and
(ii) depositing on the carrier an amount in the range of from 0.001 to 5 wt. % of one or more metals chosen from the group consisting of Groups 6, 9, 10 and 11; and optionally, up to 0.5 wt. 7, of a Group 14 metal, on the basis of the total second catalyst composition.

The binder for use in the second catalyst composition is a refractory oxide. Refractory oxides which can be used as binder in said catalyst composition include alumina, silica-alumina, aluminium phosphate, silica, zirconia and titania. The binder for the second catalyst composition is preferably selected from the group consisting of silica, zirconia, alumina, silica-alumina and aluminium phosphate.

Most preferably, silica is used as a binder in the second catalyst composition. The silica may be naturally occurring silica or may be in the form of a gelatinous precipitate, sol or gel. The form of silica is not limited and the silica may be in any of its various forms: crystalline silica, vitreous silica or amorphous silica. The term amorphous silica encompasses the wet process types, including precipitated silicas and silica gels, or pyrogenic or fumed silicas. Silica sols or colloidal silicas are non-settling dispersions of amorphous silicas in a liquid, usually water, typically stabilized by anions, cations, or non-ionic materials.

The silica binder preferably is a mixture of two silica types, most preferably a mixture of a powder form silica and a silica sol. Conveniently powder form silica has a B.E.T. surface area in the range of from 50 to 1000 m²/g; and a mean particle size in the range of from 0.002 to 200 μm, preferably in the range of from 2 to 100 μm, more preferably 2 to 60 μm, especially 2 to 10 μm as measured by ASTM C 690-1992 or ISO 8130-1.

Suitable binder materials that may be conveniently used are those available under the trade designation “Sipernat” from Evonik and “Levasil” and “Bindzil” from Nouryon.

Where the binder mixture comprises powder form silica and a silica sol, then the two components may be present in a weight ratio of powder form to sol form in the range of from 1:1 to 10:1, preferably 2:1 to 5:1, more preferably from 2:1 to 3:1. The binder may also consist essentially of just the powder form silica.

Where solely a powder form of silica is used as a binder in the second catalyst composition of the present invention, preferably a small particulate form is utilized, which has a mean particle size in the range of from 2 to 10 μm as measured by ASTM C 690-1992. An additional improvement in carrier strength is found with such materials. Suitable small particulate forms are available from Evonik under the trade name “Sipernat 50”.

Preferably the silica component is used as pure silica and not in combination with other refractory oxide components. It is most preferred that the silica and indeed the carrier for use in the second catalyst composition, is essentially free of any other inorganic oxide binder material, and especially is free of alumina. At most only a maximum of 2 wt. % alumina, based on the total refractory oxide binder, is present.

The second catalyst composition preferably comprises in the range of from 20 to 90 wt. % of the afore-mentioned refractory oxide binder in combination with in the range of from 10 to 80 wt. % of zeolite selected from one or more of ZSM-5, ferrierite, ZSM-11, ZSM-12 and EU-1, more specifically in the range of from 40 to 90 wt. % of the afore-mentioned refractory oxide binder in combination with in the range of from 10 to 60 wt. % of zeolite selected from one or more of ZSM-5, ferrierite, ZSM-11, ZSM-12 and EU-1, even more specifically in the range of from 50 to 90 wt. % of the afore-mentioned refractory oxide binder in combination with in the range of from 10 to 50 wt. % of zeolite selected from one or more of ZSM-5, ferrierite, ZSM-11, ZSM-12 and EU-1, most specifically 60 to 90 wt. % of the afore-mentioned refractory oxide binder in combination with in the range of from 10 to 40 wt. % of zeolite selected from one or more of ZSM-5, ferrierite, ZSM-11, ZSM-12 and EU-1, all based on the total weight of the carrier. The preferred zeolite in the afore-mentioned combinations is ZSM-5.

The zeolites and binders used in each of the first and second catalyst compositions may be shaped into any convenient form such as powders, extrudates, pills and granules. Preference is given to shaping by extrusion.

Generally, to prepare extrudates, commonly the required zeolite(s) will be combined with the binder composition and, if necessary, a peptizing agent, and mixed to form a dough or thick paste. The peptizing agent may be any material that will change the pH of the mixture sufficiently to induce deagglomeration of the solid particles. Peptizing agents are well known and encompass organic and inorganic acids, such as nitric acid, and alkaline materials such as ammonia, ammonium hydroxide, alkali metal hydroxides, preferably sodium hydroxide and potassium hydroxide, alkali earth hydroxides and organic amines, e.g. methylamine and ethylamine. Ammonia is a preferred peptizing agent and may be provided in any suitable form, for example via an ammonia precursor. Examples of ammonia precursors are ammonium hydroxide and urea. It is also possible for the ammonia to be present as part of the silica component, particularly where a silica sol or ZSM-5 zeolite is used, though additional ammonia may still be needed to impart the appropriate pH change. The amount of ammonia present during extrusion has been found to affect the pore structure of the extrudates which may provide advantageous properties. Suitably the amount of ammonia present during extrusion may be in the range of from 0 to 5 wt. % based on the total dry mixture, preferably 0 to 3 wt. %, more preferably 0 to 1.9 wt. %, on dry basis.

The zeolite(s) present in each of the first catalyst and second catalyst compositions have crystallite size and SAR characteristics similar to those of the zeolites used as starting compounds in their preparation. Therefore, the preferences for said properties of the zeolites which are part of catalyst compositions also apply to zeolites used in preparing the catalyst compositions.

Passivation is a treatment typically conducted on carrier compositions prior to doping with metals to form a catalyst composition. Passivation treatments may be conducted on a carrier composition before and/or after extrusion as hereinbefore described. Without being bound by theory, passivation typically serves to modify the surface of a zeolite in order to enhance its shape selectivity. Often, passivation involves deposition of silica on the zeolite surface by a silica source, for example by using organosilanes, tetraethoxy ortho silicate (TEOS) and ammonium hexafluorosilicate (AHS). Hence, optionally a passivation treatment may be conducted on the first and/or second catalyst compositions.

However, it has been surprisingly found in the present invention that it is not necessary to conduct a separate passivation treatment with a silica source on the first catalyst composition. Hence, in a preferred embodiment of the present invention, the first catalyst composition does not undergo a separate passivation treatment with a silica source.

The first and second catalyst compositions each comprise in the range of from 0.001 to 5 wt. % of one or more metals chosen from the group consisting of Groups 6, 9 and 10 and optionally a metal chosen from Group 14 in an amount up to 0.5 wt. %, on the basis of total catalyst composition. Preferably, the metal of Group 6, 9, 10 or 11 is chosen from the group consisting of tungsten, molybdenum, cobalt, nickel, palladium, platinum and silver while the metal of Group 14 is chosen from lead and tin.

More preferably, the first catalyst composition comprises in the range of from 0.001 to 0.1 wt. %, most preferably in the range of from 0.01 to 0.05 wt. %, of silver, platinum and/or palladium, most preferably platinum, based on amount of metal on total amount of catalyst and the second catalyst composition comprises in the range of from 0.001 to 0.1 wt. %, most preferably in the range of from 0.01 to 0.05 wt. %, of silver, platinum, palladium, rhenium, molybdenum, nickel and/or tungsten. Additionally, each of the first and the second catalyst compositions can contain one or more further metals, most preferably tin.

In a preferred embodiment of the present invention, the first catalyst composition, as hereinbefore described, comprises in the range of from 0.001 to 5 wt. %, preferably in the range of from 0.001 to 0.1 wt. %, most preferably in the range of from 0.01 to 0.05 wt. %, of one or more metals chosen from silver, platinum and/or palladium and optionally tin in an amount up to 0.5 wt. %, on the basis of total catalyst composition. In a particularly preferred embodiment, said first catalyst composition is used in a catalyst system further comprising a second catalyst composition, as hereinbefore described, which second catalyst composition comprises in the range of from 0.001 to 5 wt. %, preferably in the range of from 0.001 to 0.1 wt. %, most preferably in the range of from 0.01 to 0.05 wt. %, of silver, platinum, palladium, rhenium, molybdenum, nickel and/or tungsten and optionally tin in an amount up to 0.5 wt. %, on the basis of total catalyst composition.

In a most preferred embodiment, the alkylaromatic conversion catalyst system of the present invention comprises,

(a) a first catalyst composition comprising,
(i) a carrier which comprises in the range of from 5 to 70 wt. of a binder composition prepared from a mixture comprising one or more oligomerized alkoxy silicates and one or more hydrolyzing agents; and in the range of from 30 to 95 wt. % of a ZSM-5 zeolite, based on the total weight of the carrier;
(ii) an amount in the range of from 0.001 to 5 wt.
preferably in the range of from 0.001 to 0.1 wt. %, most preferably in the range of from 0.01 to 0.05 wt. %, of one or more metals chosen from the group consisting of platinum and palladium; and optionally,
(iii) up to 0.5 wt. % of tin, on the basis of the total first catalyst composition; and
(b) a second catalyst composition comprising,
(i) a carrier which comprises in the range of from 20 to 90 wt. % of refractory oxide binder and in the range of from 10 to 80 wt. of zeolite selected from one or more of ZSM-5, ferrierite, ZSM-11, ZSM-12 and EU-1, based on the total weight of the carrier;
(ii) an amount in the range of from 0.001 to 5 wt. %, preferably in the range of from 0.001 to 0.1 wt. %, most preferably in the range of from 0.01 to 0.05 wt. %, of one or more metals chosen from the group consisting of platinum and palladium; and optionally,
(iii) up to 0.5 wt. % of tin, on the basis of the total second catalyst composition.

The metals emplacement onto the carrier in each of the first and second catalyst compositions may be effected by methods usual in the art. The metals can be deposited onto the carriers prior to shaping, but it is preferred to deposit them onto a shaped carrier.

Pore volume impregnation of the metals from a metal salt solution is a very suitable method of metals emplacement onto a shaped carrier. The metal salt solutions may have a pH in the range of from 1 to 12. The platinum salts that may conveniently be used are chloroplatinic acid and ammonium stabilized platinum salts. If tin is present, the tin preferably is added as a salt selected from the group consisting of stannous (II) chloride, stannic (IV) chloride, stannous sulphate, stannous oxalate and stannous acetate.

If different metals are deposited on the carriers, the metals may be impregnated onto the shaped carriers either sequentially or simultaneously. Where simultaneous impregnation is utilized the metal salts used must be compatible and not hinder the deposition of the metals.

After shaping of the carriers, and also after metals impregnation, the carriers/catalysts are typically dried, and calcined. Drying temperatures are suitably in the range of from 20 to 200° C.; drying times are suitably from 0.5 to 5 hours. Calcination temperatures are very suitably in the range of from 200 to 800° C., preferably 300 to 750° C. For calcination of the carriers, a relatively short time period is required, for example 0.5 to 3 hours. For calcination of the catalyst compositions, it may be necessary to employ controlled temperature ramping at a low rate of heating to ensure the optimum dispersion of the metals. Such calcination may require from 5 to 20 hours.

Prior to use, it is generally necessary to ensure that the metals on the catalyst compositions are in metallic (and not oxidic) form. Accordingly, it is useful to subject the compositions to reducing conditions, which are, for example, heating in a reducing atmosphere, such as in hydrogen optionally diluted with an inert gas, or mixture of inert gases, such as nitrogen and carbon dioxide, at a temperature in the range of from 150 to 600° C. for from 0.5 to 5 hours.

In a preferred embodiment of the present invention, the weight ratio of the first catalyst composition to the second catalyst composition in the alkylaromatic conversion catalyst system is in the range of from 25:75 to 75:25, more preferably in the range of from 40:60 to 60:40.

In a preferred embodiment of the present invention, there is provided an alkylaromatic conversion catalyst system comprising,

(a) a first catalyst composition comprising,
(i) a carrier which comprises in the range of from 5 to 70 wt. % of a binder composition prepared from a mixture comprising one or more oligomerized alkoxy silicates and one or more hydrolyzing agents; and in the range of from 30 to 95 wt. % of a ZSM-5 zeolite, based on the total weight of the carrier;
(ii) an amount in the range of from 0.001 to 5 wt. % of one or more metals chosen from the group consisting of Groups 6, 9 and 10; and optionally,
(iii) up to 0.5 wt. % of a Group 14 metal, on the basis of the total first catalyst composition; and
(b) a second catalyst composition comprising,
(i) a carrier which comprises in the range of from 20 to 90 wt. % of refractory oxide binder and in the range of from 10 to 80 wt. % of ZSM-5 zeolite, based on the total weight of the carrier;
(ii) an amount in the range of from 0.001 to 5 wt. % of one or more metals chosen from the group consisting of Groups 6, 9 and 10; and optionally,
(iii) up to 0.5 wt. % of a Group 14 metal, on the basis of the total second catalyst composition.

In a most preferred embodiment of the present invention, there is provided an alkylaromatic conversion catalyst system comprising,

(a) a first catalyst composition comprising,
(i) a carrier which comprises in the range of from 5 to 70 wt. % of a binder composition prepared from a mixture comprising one or more oligomerized alkoxy silicates and one or more hydrolyzing agents; and in the range of from 30 to 95 wt. % of a ZSM-5 zeolite, based on the total weight of the carrier;
(ii) an amount in the range of from 0.001 to 5 wt. % of one or more metals chosen from the group consisting of platinum and palladium; and optionally,
(iii) up to 0.5 wt. % of tin, on the basis of the total first catalyst composition; and
(b) a second catalyst composition comprising,
(i) a carrier which comprises in the range of from 20 to 90 wt. % of refractory oxide binder and in the range of from 10 to 80 wt. % of ZSM-5 zeolite, based on the total weight of the carrier;
(ii) an amount in the range of from 0.001 to 5 wt. % of one or more metals chosen from the group consisting of platinum and palladium; and optionally,
(iii) up to 0.5 wt. % of tin, on the basis of the total second catalyst composition.

In the alkylaromatic conversion catalyst system of the present invention, the first and second catalyst compositions can be stacked in a layered configuration, they can be provided in the same bed but still in separate layers, in separate zones/sections or in separate beds, or each of the catalysts may be provided in multiple beds, and the catalyst may be provided in the same reactor, in separate reactors or each in multiple reactors, optionally separated by an intermediate section. For the avoidance of doubt, in the afore-mentioned arrangements, the order of the first and second catalysts is not limited and the hydrocarbon feedstock may first come into contact with either the first or the second catalyst composition.

Alternatively, the first and second catalyst compositions may be mixed together in some or all of the catalyst bed. Thus, one embodiment, the catalyst bed may be made up of a uniform mixture of the first and second catalyst compositions throughout the entirety of the catalyst bed. In another embodiment, the catalyst bed may comprise distinct layers of the first and second catalyst compositions and/or one or more mixtures of the first and second catalysts in differing proportions and at different locations in the catalyst bed.

It is preferred in the alkylaromatic conversion catalyst system of the present invention that the second catalyst composition is located downstream from the first catalyst composition with regard to the direction of feedstock flow, such that the hydrocarbon feedstock will first come into contact with the first catalyst composition.

It is particularly preferred that the first and second catalyst compositions are located in the same reactor in an arrangement wherein the second catalyst composition is located downstream from the first catalyst composition with regard to the direction of feedstock flow, such that the hydrocarbon feedstock will first come into contact with the first catalyst composition.

For example, any type of reactor may be loaded in such a way that the hydrocarbon feedstock will first pass the first catalyst composition and the converted feedstock and any unreacted feedstock will then subsequently pass the second catalyst composition. When, for example, an industrial reactor is operated in a top-down flow, the preparation of the catalyst system generally will comprise loading the reactor with the first and second catalyst compositions in such a way that the second catalyst composition is positioned in the lower sections of the reactor and loading the first catalyst composition above that.

For commercial units various loading techniques are available which are often executed by specialised companies (like the Dutch company “Mourik”).

After loading, the catalyst compositions may undergo an optional pre-treatment in the presence of hydrogen at temperatures in the range of from 300 to 500° C., prior to addition of the hydrocarbon feedstock for use in an ethylbenzene dealkylation process. Additionally, to achieve a stable performance, the catalyst compositions may undergo an ageing step by exposing the system to severe reaction conditions such as temperatures in the range of from 380 to 480° C. and/or low hydrogen:hydrocarbon feedstock molar ratios of less than 1.5, prior to the actual reaction conditions.

The present invention further provides an ethylbenzene dealkylation process which comprises contacting a hydrocarbon feedstock which comprises ethylbenzene in the presence of hydrogen with an alkylaromatic conversion catalyst system as hereinbefore described.

In a preferred embodiment, the process of the present invention comprises contacting a hydrocarbon feedstock which comprises ethylbenzene in the presence of hydrogen with an alkylaromatic conversion catalyst system comprising,

(a) a first catalyst composition comprising,
(i) a carrier which comprises in the range of from 5 to 70 wt. % of a binder composition prepared from a mixture comprising one or more oligomerized alkoxy silicates and one or more hydrolyzing agents; and in the range of from 30 to 95 wt. % of a ZSM-5 zeolite, based on the total weight of the carrier;
(ii) an amount in the range of from 0.001 to 5 wt. % of one or more metals chosen from the group consisting of Groups 6, 9, 10 and 11; and optionally,
(iii) up to 0.5 wt. % of a Group 14 metal, on the basis of the total first catalyst composition; and
(b) a second catalyst composition comprising,
(i) a carrier which comprises in the range of from 20 to 90 wt. % of refractory oxide binder and in the range of from 10 to 80 wt. % of zeolite selected from one or more of ZSM-5, ferrierite, ZSM-11, ZSM-12 and EU-1, based on the total weight of the carrier;
(ii) an amount in the range of from 0.001 to 5 wt. % of one or more metals chosen from the group consisting of Groups 6, 9, 10 and 11; and optionally,
(iii) up to 0.5 wt. % of a Group 14 metal, on the basis of the total second catalyst composition.

In a particularly preferred embodiment, the process of the present invention comprises contacting a hydrocarbon feedstock which comprises ethylbenzene in the presence of hydrogen with:

(a) a first catalyst composition comprising,
(i) a carrier which comprises in the range of from 5 to 70 wt. % of a binder composition prepared from a mixture comprising one or more oligomerized alkoxy silicates and one or more hydrolyzing agents; and in the range of from 30 to 95 wt. % of a ZSM-5 zeolite, based on the total weight of the carrier;
(ii) an amount in the range of from 0.001 to 5 wt. % of one or more metals chosen from the group consisting of Groups 6, 9 and 10; and optionally,
(iii) up to 0.5 wt. % of a Group 14 metal, on the basis of the total first catalyst composition; and
(b) a second catalyst composition comprising,
(i) a carrier which comprises in the range of from 20 to 90 wt. % of refractory oxide binder and in the range of from 10 to 80 wt. % of ZSM-5 zeolite, based on the total weight of the carrier;
(ii) an amount in the range of from 0.001 to 5 wt. % of one or more metals chosen from the group consisting of Groups 6, 9 and 10; and optionally,
(iii) up to 0.5 wt. % of a Group 14 metal, on the basis of the total second catalyst composition.

Particularly preferred alkylaromatic conversion catalyst systems for use in the ethylbenzene dealkylation process of the present invention have been described hereinbefore.

The hydrocarbon feedstock for use in the process of the present invention most suitably originates from a reforming unit or naphtha pyrolysis unit or is the effluent of a xylene isomerisation or transalkylation unit. After distillation and para-xylene extraction, such hydrocarbon feedstock usually comprises hydrocarbons containing of from 7 to 9 carbon atoms, and in particular, one or more of o-xylene, m-xylene, and p-xylene in addition to ethylbenzene. Typically, the xylenes will not be in a thermodynamic equilibrium, and the content of p-xylene will accordingly be lower than that of the other isomers.

Generally, the amount of ethylbenzene in the hydrocarbon feedstock is in the range of from 0.1 to 50 wt. % and the total xylene content is typically of from 20 to 99.9 wt. %, each based on total amount of hydrocarbon feedstock. More specifically, the total xylene content is typically at least 30 wt. %.

The hydrocarbon feedstock is contacted with the catalyst in the presence of hydrogen. This may be carried out in a fixed bed system. Such a system may be operated continuously or in batch fashion. Preference is given to continuous operation in a fixed bed system. The alkylaromatic conversion catalyst system may be used in one reactor or in several separate reactors in series or operated in a swing system to ensure continuous operation during catalyst change-out.

The ethylbenzene dealkylation process is suitably carried out at a temperature in the range of from 300 to 500° C., a pressure in the range of from 0.1 to 50 bar (10 to 5,000 kPa), using a weight hourly space velocity of in the range of from 0.5 to 20 g hydrocarbon feedstock/g catalyst/hour.

A partial pressure of hydrogen in the range of from 0.05 to 30 bar (5 to 3,000 kPa) is generally used. The hydrogen to feed molar ratio is in the range of from 0.5 to 100, generally from 1 to 10 mol/mol.

In a preferred embodiment of the ethylbenzene dealkylation process, the weight hourly space velocity is of from 7 to 15 g hydrocarbon feedstock/g catalyst/hour, the pressure is in the range of from 5 to 25 bar and the hydrogen/hydrocarbon feedstock molar ratio is in the range of from 1 to 5.

The present invention will now be illustrated by the following Examples.

### Examples

### Catalyst Manufacture

### Catalyst A

8.78 g of demineralized water and 0.43 g ammonia were added to a mix of 22.2 g large crystallite ZSM-5 (crystallite size (as determined with SEM) between 1-5 micrometer with 4 micrometer on average), 2.09 g of “Sipernat 50” binder, 6.25 g of “Levasil” binder.

The resulting material was mixed, mulled and extruded.

Afterwards the extrudates were treated with 15% steam in air at 750° C. for 50 minutes, ion exchanged with 1 M NH₄Ac to remove any residual Na followed by calcination at 550° C. for 1 hour, and subsequently incipient wetness impregnated with hydrochloroplatinic acid dissolved in 0.7 M hydrochloric acid to obtain 0.02 wt. % Pt.

After impregnation the catalyst was dried and calcined at 550° C. to obtain the final catalyst.

### Catalyst B

0.09 g of ammonia (28%) dissolved in 15.4 g demineralized water was taken to a pH of 8 with ammonia and added to 18.75 g Ethyl Silicate 40 (ETS-40) binder (available from Dr. Khan Industrial Consultants) and stirred for 30 minutes at room temperature. The resulting liquid was added to 44 g large crystallite ZSM-5 zeolite (crystallite size (as determined with SEM) between 1-5 micrometer with 4 micrometer on average).

The resulting material was mixed, mulled and extruded.

Subsequently the extrudates were dried at 120° C. and calcined at 550° C. for 1 hour.

Afterwards the extrudates were treated with 15% steam in air at 750° C. for 50 minutes, ion exchanged with 1 M NH₄Ac to remove any residual Na followed by calcination at 550° C. for 1 hour, and subsequently incipient wetness impregnated with hydrochloroplatinic acid dissolved in 0.7 M hydrochloric acid to obtain 0.02 wt. % Pt.

After impregnation the catalyst was dried and calcined at 550° C. to obtain the final catalyst.

### Catalyst C

9.34 g of demineralized water and 1.53 g ammonia were added to a mix of 13.79 g small crystallite (approx. 50 nm) ZSM-5, 6.56 g of “Sipernat 50” binder, 9.76 g of “Levasil” binder.

The resulting material was mixed, mulled, extruded and calcined at 575° C. for 1 hour.

Afterwards, the extrudates were ion exchanged with 1 M NH₄Ac to remove any residual Na followed by calcination at 550° C. for 1 hour, and subsequently incipient wetness impregnated with hydrochloroplatinic acid and stannous chloride dissolved in 0.7 M hydrochloric acid to obtain 0.025 wt. % Pt and 0.1 wt. % Sn.

After impregnation the catalyst was dried calcined at 550° C. for 1 hour to obtain the final catalyst.

### Preparation of Catalyst Systems

Table 1 summarises the individual catalysts that were prepared.

Catalyst compositions were used alone and/or in various combinations for subsequent testing. When used in combination, a top catalyst (Catalysts A and B) was loaded on top of a bottom catalyst (Catalyst C) in a 50:50 gravimetric ratio. The reactor flow went from top to bottom.

TABLE 1

| | Zeolite | | | | | | | |
|---|---|---|---|---|---|---|---|---|
| | | Zeolite | Carrier | | | | | |
| | | Silica to | Zeolite | | | | | |
| | Zeolite | Alumina | (wt. %) | | | Zeolite | Carrier Dopants | |
| | Crystallite | Ratio | in | Binder in Carrier | | Treated? | Pt | Sn |
| Catalyst | Size (nm) | (SAR) | Carrier | Type | (wt. %) | Yes/No | (wt. %) | (wt. %) |
| A | 1000-5000 | 60 | 85 | “Sipernat”/ | 7.5/7.5 | Yes | 0.02 | — |
| | | | | “Levasil” | | | | |
| B | 1000-5000 | 60 | 85 | ETS-40 | 15 | Yes | 0.02 | — |
| C | 3-100 | 60 | 30 | “Sipernat”/ | 50/20 | No | 0.025 | 0.1 |
| | | | | “Levasil” | | | | |

### Testing of Catalyst Systems

Testing was conducted under catalytic testing conditions that mimics typical industrial application conditions for ethylbenzene dealkylation. This activity test uses a feed having the composition summarized in

TABLE 2
Composition of the feed used in the activity testing

| Component | (wt. % in feed composition) |
|---|---|
| Ethylbenzene (EB) | 13.0 |
| p-Xylene (pX) | 4.2 |
| o-Xylene (oX) | 17.1 |
| m-Xylene (mX) | 63.8 |
| C₇-C₉-paraffins | 1.9 |
| C₉₊ aromatics | 0.00 |
| Total | 100.00 |

Other Feed Characteristics

| | |
|---|---|
| C₈ aromatics sum (wt. %) | 98.1 |
| EB in C8 aromatics feed (wt. %) | 13.3 |
| pX (wt. %) in xylenes in feed | 5.0 |
| oX (wt. %) in xylenes in feed | 20.0 |
| mX (wt. %) in xylenes in feed | 75.0 |

The activity test is performed in a fixed bed unit with online GC analysis once the catalyst is in its reduced state, which is achieved by exposing the dried and calcined catalyst to atmospheric hydrogen (>99% purity) at 450° C. for 1 hour.

After reduction, the reactor is cooled down to 380° C., pressurized to 1.2 MPa and the feed is introduced at a weight hourly space velocity of 12 g feed/g catalyst/hour and a hydrogen to feed ratio of 2.5 mol·mol⁻¹. Subsequently, the temperature is increased to 450° C. and the weight hourly space velocity decreased to 10 g feed/g catalyst/hour and the hydrogen to feed ratio to 1 mol·mol⁻¹. This step contributes to enhanced catalyst aging, and therefore allows comparison of the catalytic performance at stable operation. After 24 hours the conditions are switched to the actual operating conditions.

In the present case, a weight hourly space velocity of 12 g feed/g catalyst/hour, hydrogen to feed ratio of 2.5 mol·mol⁻¹ and a total system pressure of 1.2 MPa was used. The temperature was varied between 340 and 380° C.

The performance characteristics including the products obtained are shown in Table 3 below. Comparisons are made at 355° C.

Ethylbenzene conversion (EB conversion) is the weight percent of ethylbenzene converted by the catalyst into benzene and ethylene, or other molecules. It is defined as wt. % ethylbenzene in feed minus wt. % ethylbenzene in product divided by wt. % ethylbenzene in feed times 100%.

PX/X is the percentage of para-xylene (pX) in xylenes (pX, meta-xylene (mX) and ortho-xylene (oX)) in the effluent. The equilibrium concentration at 355° C. is assumed to be 24.2%.

The formation of C₉ aromatic components, such as trimethylbenzene (TMB) and methylethylbenzene (MEB) and C₁₀ or larger aromatic components are unwanted as they form at the expense of preferred products such as p-xylene and benzene.

TABLE 3

| | Catalyst System* | | | | | | | |
|---|---|---|---|---|---|---|---|---|
| | | | Weight Ratio | Results | | | | |
| | 1^{st} | 2^{nd} | of 1^{st}:2^{nd} | EB | | TMB in | MEB in | C₁₀₊ in |
| | Catalyst | Catalyst | Catalyst | conversion, | PX/X | product, | product, | product, |
| Example | Composition | Composition | Compositions | wt. % | % | wt. % | wt. % | wt. % |
| 1 | A | — | 50:0 | 34.8 | 16.0 | 0.07 | 0.16 | 0.13 |
| (comp. ) | | | | | | | | |
| 2 | B | — | 50:0 | 48.2 | 12.9 | 0.03 | 0.12 | 0.09 |
| (comp. ) | | | | | | | | |
| 3 | — | C | 0:50 | 13.9 | 24.0 | 0.27 | 0.14 | 0.17 |
| (comp. ) | | | | | | | | |
| 4 | A | C | 50:50 | 50.6 | 24.2 | 0.17 | 0.31 | 0.15 |
| (comp. ) | | | | | | | | |
| 5 | B | C | 50:50 | 63.5 | 24.2 | 0.09 | 0.23 | 0.09 |
| (inv.) | | | | | | | | |

*Where only a 1^{st} or 2^{nd} Catalyst Composition is indicated, then the catalyst composition was a single catalyst bed.

### Discussion of Results

The above experimental results show that the combination of catalysts according to the present invention (Example 5) allows a high dealkylation activity of an alkylaromatic-containing hydrocarbon feedstock while simultaneously isomerizing xylenes such that a product is obtained which is relatively high in para-xylene and also low in unwanted heavier components such as TMB, MEB and C₁₀₊ components.

The results obtained for Example 5, utilizing a combination of catalysts as described in the present invention, are surprisingly better than the results obtained in Examples 1-3 for various single catalysts (Catalysts A-C) and are also better than for Example 4 which utilized similar zeolite contents but different silica binders for the first catalyst.

1. An alkylaromatic conversion catalyst system comprising,
(a) a first catalyst composition comprising,
(i) a carrier which comprises in the range of from 5 to 70 wt. % of a binder composition prepared from a mixture comprising one or more oligomerized alkoxy silicates and one or more hydrolyzing agents; and in the range of from 30 to 95 wt. % of a ZSM-5 zeolite, based on the total weight of the carrier;
(ii) an amount in the range of from 0.001 to 5 wt. % of one or more metals chosen from the group consisting of Groups 6, 9, 10 and 11; and optionally,
(iii) up to 0.5 wt. % of a Group 14 metal, on the basis of the total first catalyst composition; and
(b) a second catalyst composition comprising,
(i) a carrier which comprises in the range of from 20 to 90 wt. % of refractory oxide binder and in the range of from 10 to 80 wt. % of zeolite selected from one or more of ZSM-5, ferrierite, ZSM-11, ZSM-12 and EU-1, based on the total weight of the carrier;
(ii) an amount in the range of from 0.001 to 5 wt. % of one or more metals chosen from the group consisting of Groups 6, 9, 10 and 11; and optionally,
(iii) up to 0.5 wt. % of a Group 14 metal, on the basis of the total second catalyst composition.

2. Alkylaromatic conversion catalyst system as claimed in claim 1, wherein the second catalyst composition is located downstream from the first catalyst composition.

3. Alkylaromatic conversion catalyst system as claimed in claim 1, wherein the ZSM-5 zeolite in the first catalyst composition has a crystallite size of from 200 to 5000 nm and a molar silica to alumina ratio in the range of from 20 to 100.

4. Alkylaromatic conversion catalyst system as claimed in claim 1, wherein the binder composition in the first catalyst composition is prepared from a mixture comprising one or more oligomerized alkoxy silicates having the following general formula:—
where n=1, 2, or higher and R is a linear alkyl group with 1 to 6 carbon atoms and one or more hydrolyzing agents selected from water, aqueous solutions of metal nitrate salts, aqueous and organic solutions of acids, and bases.

5. Alkylaromatic conversion catalyst system as claimed in claim 1, wherein the carrier in the first catalyst composition is composed of in the range of from 5 to 20 wt. % of the binder composition, and in the range of from 80 to 95 wt. % ZSM-5 zeolite, based on the total weight of the carrier.

6. Alkylaromatic conversion catalyst system as claimed in claim 1, wherein the ZSM-5 zeolite in the first catalyst composition has a crystallite size of from 1000 to 5000 nm.

7. Alkylaromatic conversion catalyst system as claimed in claim 1, wherein the weight ratio of the first catalyst composition to the second catalyst composition is in the range of from 25:75 to 60:40.

8. A method of making an alkylaromatic conversion catalyst system as claimed in claim 1, wherein said method comprises making the first catalyst composition by:—
(i) preparing a binder composition from a mixture comprising one or more oligomerized alkoxy silicates and one or more hydrolyzing agents;
(ii) preparing a carrier comprising in the range of from 5 to 70 wt. % of said binder composition; and in the range of from 30 to 95 wt. % of a ZSM-5 zeolite, based on the total weight of the carrier; and
(iii) depositing on the carrier an amount in the range of from 0.001 to 5 wt. % of one or more metals chosen from the group consisting of Groups 6, 9, 10 and 11; and optionally, up to 0.5 wt. % of a Group 14 metal, on the basis of the total first catalyst composition.

9. Method as claimed in claim 8, wherein said method further comprises making the second catalyst composition by:—
(i) preparing a carrier which comprises in the range of from 20 to 90 wt. % of refractory oxide binder and in the range of from 10 to 80 wt. % of zeolite selected from one or more of ZSM-5, ferrierite, ZSM-11, ZSM-12 and EU-1, based on the total weight of the carrier; and
(ii) depositing on the carrier an amount in the range of from 0.001 to 5 wt. % of one or more metals chosen from the group consisting of Groups 6, 9, 10 and 11; and optionally, up to 0.5 wt. % of a Group 14 metal, on the basis of the total second catalyst composition.

10. Method as claimed in claim 8, wherein the ZSM-5 zeolite in the first catalyst composition undergoes a dealumination treatment.

11. An ethylbenzene dealkylation process which comprises contacting a hydrocarbon feedstock which comprises ethylbenzene in the presence of hydrogen with an alkylaromatic conversion catalyst system as claimed in claim 1.

12. Ethylbenzene dealkylation process according to claim 11, in which process the weight hourly space velocity is of from 7 to 15 g hydrocarbon feedstock/g catalyst/hour, the pressure is in the range of from 5 to 25 bar and the hydrogen/hydrocarbon feedstock molar ratio is of from 1 to 5.
